(19) United States
(12) Patent Application Publication
(10) Pub. No.: US 2007/0067022 A1
(43) Pub. Date: Mar. 22, 2007

(54) Implantable support frame with electrolytically removable material

(75) Inventors: Brian C. Case, Lake Villa, IL (US); Jacob A. Flagle, Indianapolis, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/511,008

(22) Filed: Aug. 28, 2006

Related U.S. Application Data
(60) Provisional application No. 60/713,769, filed on Sep. 2, 2005.

(51) Int. Cl. A61F 2/06; A61F 2/82

(52) U.S. Cl. 623/1.24; 623/1.16

(57) ABSTRACT

Endoluminally implantable medical devices having an electrolytically removable portion are provided, as well as methods pertaining to the same. The electrolytically removable portion can be dissolved within a body vessel after implantation of the medical device, by application of an electrical current from an electrode on a catheter inside the body vessel or by induction of an electrical current within the removable region. Electrolytic dissolution of the removable portion can alter the mechanical strength of the implanted frame within the body vessel. Medical devices can be an endovascular valve comprising a frame and one or more valve members adapted to regulate fluid flow in a body vessel, such as a vein.

### RELATED APPLICATIONS

This application claims the benefit of U.S. provisional patent application Ser. No. 60/713,769, filed Sep. 2, 2005 (Case et al.), which is incorporated herein by reference in its entirety.

### TECHNICAL FIELD

The present invention relates to medical devices for implantation in a body vessel. In particular, medical devices comprising electrolytically removable material are provided.

### BACKGROUND

Various implantable medical devices are advantageously inserted within various body vessels, for example from an implantation catheter. Minimally invasive techniques and instruments for placement of intraluminal medical devices have been developed to treat and repair such undesirable conditions within body vessels, including treatment of venous valve insufficiency. Intraluminal medical devices can be deployed in a vessel at a point of treatment, the delivery device withdrawn from the vessel, and the medical device retained within the vessel to provide sustained improvement in vascular valve function. For example, implantable medical devices can function as a replacement venous valve, or restore native venous valve function by bringing incompetent valve leaflets into closer proximity. Such devices can comprise an expandable frame configured for implantation in the lumen of a body vessel, such as a vein. Venous valve devices can further comprise features that provide a valve function, such as opposable leaflets.

Implantable medical devices can comprise frames that are highly compliant, and therefore able to conform to both the shape of the lumen of a body vessel as well as respond to changes in the body vessel shape. Dynamic fluctuations in the shape of the lumen of a body vessel pose challenges to the design of implantable devices that conform to the interior shape of the body vessel. The shape of a lumen of a vein can undergo dramatic dynamic change as a result of varying blood flow velocities and volumes therethrough, presenting challenges for designing implantable intraluminal prosthetic devices that are compliant to the changing shape of the vein lumen.

Optimizing the degree to which a medical device for implantation within a body vessel is compliant to changes in the shape of the body vessel can involve consideration of various factors. For example, a medical device comprising a highly compliant frame can minimize distortion of a body vessel by being highly responsive to changes in the shape of the body vessel. For some applications, an implantable frame that can be changed by medical intervention after implantation would be useful. For example, it may be desirable to implant an implantable medical device adapted to provide a first radial strength upon implantation within a body vessel, where the medical device is adapted for later modification within the body vessel to reduce the radial strength.

For treatment of many conditions, it is desirable that implantable medical devices comprise remodelable material. Implanted remodelable material provides a matrix or support for the growth of new tissue thereon, and remodelable material is resorbed into the body in which the device is implanted. Common events during this remodeling process include: widespread neovascularization, proliferation of granulation mesenchymal cells, biodegradation/resorption of implanted remodelable material, and absence of immune rejection. By this process, autologous cells from the body can replace the remodelable portions of the medical device.

Mechanical loading of remodelable material during the remodeling process has been shown to advantageously influence the remodeling process. For example, the remodeling process of one type of remodelable material, extracellular matrix (ECM), is more effective when the material is subject to certain types and ranges of mechanical loading during the remodeling process. See, e.g., M. Chiquet, “Regulation of extracellular matrix gene expression by pressure,” Matrix Biol. 18(5), 417-426 (October 1999). Mechanical forces on a remodelable material during the remodeling process can affect processes such as signal transduction, gene expression and contact guidance of cells. See, e.g., VC Mudera et al., “Molecular responses of human dermal fibroblasts to dual cues: contact guidance and mechanical load,” Cell Motil. Cytoskeleton, 45(1):1-9 (June 2000).

Therefore, a highly compliant frame with minimal radial strength may provide inadequate mechanical loading to material attached to the frame to allow or promote certain desirable processes to occur within the attached material, such as remodeling, or within the body vessel. In some instances, frame radial strength can be a trade-off between enabling the remodeling of material attached to the frame, and minimizing the distortion or disruption of the body vessel. Electrolytically dissolvable sacrificial links have been used to detach a delivery catheter from a deployable medical device, such as an embolic coil. For example, U.S. Pat. No. 6,425,914 (Wallace et al.) describes endovascular implants that are detachable from a delivery catheter by electrolytic dissolution of an electrolytically dissolvable link. What is needed are medical devices that provide a radial strength that can be reduced by a medical intervention after implantation, for example by electrolytic dissolution of one or more removable frame portions within a body vessel using an intravascular catheter. There exists a need in the art for an implantable prosthetic device frame that is capable of balancing concerns of conforming to the shape of a body vessel lumen and providing optimal tension on a remodelable material attached to the frame.

Implantable frames with radial strength that can be altered to provide desired levels of radial strength after a desired period of implantation within a body vessel. Medical devices with a radial strength that can be altered can provide, for example, an optimal amount of tension on an attached remodelable material during the remodeling process, and then provide decreased radial strength and minimal body vessel distortion after the remodeling process is completed.

### SUMMARY

Medical devices including an implantable frame with one or more removable portions are provided. The removable portions can be dissolved or weakened by electrolysis in an aqueous environment, such as within a body vessel, by the application of electrical current to the implantable frame or portions thereof. An intraluminal conducting member, such as a catheter adapted or configured for electrical conduction, can be positioned in electrically conducting orientation with respect to a portion of the implanted frame within a body vessel. Subsequent application of an electrical current from the intraluminal conducting member to the implanted frame can dissolve the removable portion in situ, thereby altering the mechanical properties or configuration of the implanted frame. Dissolution of the removable portion of the implanted frame can increase the flexibility of the implanted frame, or alter the configuration of the frame to permit removal of the frame using a catheter within the body vessel.

A medical device preferably comprises an implantable frame that is expandable from a compressed delivery configuration to an expanded deployment configuration. In one aspect, a medical device comprises a self-expanding material. In another aspect, a medical device is expanded using a balloon catheter. Medical devices are preferably delivered intraluminally, for example using various types of delivery catheters, and be expanded by conventional methods such as balloon expansion or self-expansion. A medical device can optionally comprise means for orienting the frame within a body lumen. For example, the frame can comprise a marker, or a delivery device comprising the frame can provide indicia relating to the orientation of the frame within the body vessel.

A particularly preferred medical device is an intraluminally implantable prosthetic valve comprising an expandable support frame having a detachable portion and at least one leaflet comprising a remodelable material attached to the support frame. However, any suitable medical device comprising an implantable frame can be used. One preferred embodiment provides an implantable prosthetic valve comprising a support frame with at least one electrolytic dissolution region. The support frame is desirably configured to initially provide a first mechanical load across a valve leaflet comprising a remodelable material attached to the support frame. Preferably, weakening or dissolution of the electrolytic dissolution region results in a reduction in the mechanical load across the valve leaflet.

Some embodiments provide methods of treating a subject, which can be animal or human, comprising the step of implanting one or more medical devices as described herein. In some embodiments, methods of treating may also include the step of delivering a medical device to a point of treatment in a body vessel, or deploying a medical device at the point of treatment. Some methods further comprise the step of implanting one or more medical devices each comprising a frame attached to one or more valve members, as described herein. Methods for treating certain conditions are also provided, such as venous valve insufficiency, varicose veins, esophageal reflux, restenosis or atherosclerosis.

The implantable frame can perform any desired function within the body vessel, but is preferably a support frame attached to a remodelable material as part of an implantable prosthetic valve. A medical device can be delivered to any suitable body vessel, such as a vein, artery, biliary duct, ureteral vessel, body passage or portion of the alimentary canal. In some embodiments, medical devices having a frame with a compressed delivery configuration with a suitably low profile, small collapsed diameter and great flexibility, may be able to navigate small or tortuous paths through a variety of body vessels. A low-profile medical device may also be useful in coronary arteries, carotid arteries, vascular aneurysms, and peripheral arteries and veins (e.g., renal, iliac, femoral, popliteal, subclavian, aorta, intercranial, etc.). Other nonvascular applications include gastrointestinal, duodenum, biliary ducts, esophagus, urethra, reproductive tracts, trachea, and respiratory (e.g., bronchial) ducts.

### BRIEF DESCRIPTION OF THE DRAWINGS

FIG. 1 is a longitudinal cross sectional view of a first frame segment including a removable portion.

FIG. 2 is a longitudinal cross sectional view of a second frame segment including a removable portion.

FIG. 3A is a side view of an implantable valve with a frame support comprising a removable portion.

FIG. 3B is an end view of the implantable valve shown in FIG. 3A in the open state.

FIG. 4 is a cutaway view of a body vessel segment, showing a catheter electrical conducting member positioned in electrically conducting contact with an implantable valve having a frame support that includes a removable portion and positioned.

FIG. 5 is a cross sectional view of the distal portion of a second catheter electrical conducting member.

### DETAILED DESCRIPTION

The following detailed description and appended drawings describe and illustrate various exemplary embodiments. Various medical devices for implantation in a body vessel, methods of making the medical devices, and methods of treatment that utilize the medical devices are provided herein.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel. An “endovascularly deployable frame” is a frame configured for implantation within a vascular body vessel. The terms “implantable frame,” “frame” and “support frame” are used interchangeable herein, unless otherwise indicated.

As used herein, “endolumenally” or “endovascularly” means placement by procedures wherein the prosthesis is translumenally advanced through the lumen of a body vessel from a remote location to a target site within the body vessel. In vascular procedures, a medical device will typically be introduced “endovascularly” using a catheter over a guidewire under fluoroscopic guidance. The catheters and guidewires may be introduced through conventional access sites to the vascular system, such as through the femoral artery, or brachial and subclavian arteries, for access to the coronary arteries.

The medical devices described herein are preferably radially expandable. By “radially expandable,” it is meant that the body segment can be converted from a small diameter configuration (used for endolumenal placement) to a radially expanded, usually cylindrical, configuration which is achieved when the medical device is implanted at the desired target site. A medical device can be radially expanded by any suitable mechanism.

As used herein, “bioabsorbable polymer” refers to a polymer or copolymer which is dissipated within the body.

A “biocompatible” material is a material that is compatible with living tissue or a living system by not being undesirably toxic or injurious and not causing immunological rejection.

“Non-bioabsorbable” material refers to a material, such as a polymer or copolymer, which remains in the body without substantial dissipation.

The recitation of a “proximal” or “distal” direction are provided as directions relative to each other, not with respect to the body vessel. Any suitable orientation or direction may correspond to a “proximal” or “distal” direction, unless otherwise indicated. The medical devices of the embodiments described herein may be oriented in any suitable absolute orientation with respect to a body vessel. In a vein, antegrade fluid flow proceeds toward the heart. Antegrade fluid flow through a valve implanted within a vein desirably proceeds from the proximal end to the distal end of the valve.

“Radial strength” (also called “hoop strength”) refers to the ability of a medical device to resist external circumferential pressure directed radially inward toward the center of a cross sectional area of the medical device, as measured by the change in diameter of the medical device as a function of inward circumferential pressure. A reduction in radial strength over time is measured by comparing the frame displacement in response to a force applied to the frame in the same manner at two different points in time. Preferably, the radial strength is measured using a Radial Force Gauge. “Radial expansion force” refers to the outward radial force exerted by the expansion of a medical device from a radially compressed configuration.

The term “removable portion” refers to a material that can be removed from a medical device within a body, preferably by electrolytic dissolution during application of sufficient current to the removable portion in an electrolytic medium.

A “mechanical load” means any force applied to a material that results in tension within the material. In preferred embodiments, a remodelable material is subject to adequate mechanical load to promote desired remodeling processes to occur.

An “electrolytic medium” refers herein to any medium that permits electrolysis of a removable frame portion to occur. Preferably, the medium is a fluid medium such as an aqueous liquid. Unless otherwise specified, the term “aqueous liquid” includes suitable saline or pH buffered environments, including phosphate buffered saline, blood or plasma.

A medical device comprising a removable portion can be endovascularly inserted into the vascular cavity. A removable portion of a frame can be dissolved electrolytically within a body vessel. Removal of one or more removable portions of an implanted frame can change the configuration or properties of the frame. For example, electrolytic dissolution of a removable portion can introduce a discontinuity, a break or a bend in an implanted frame, thereby changing the radial strength of a frame or permitting removal or movement of the frame using a catheter based device.

Removable Frame Portions

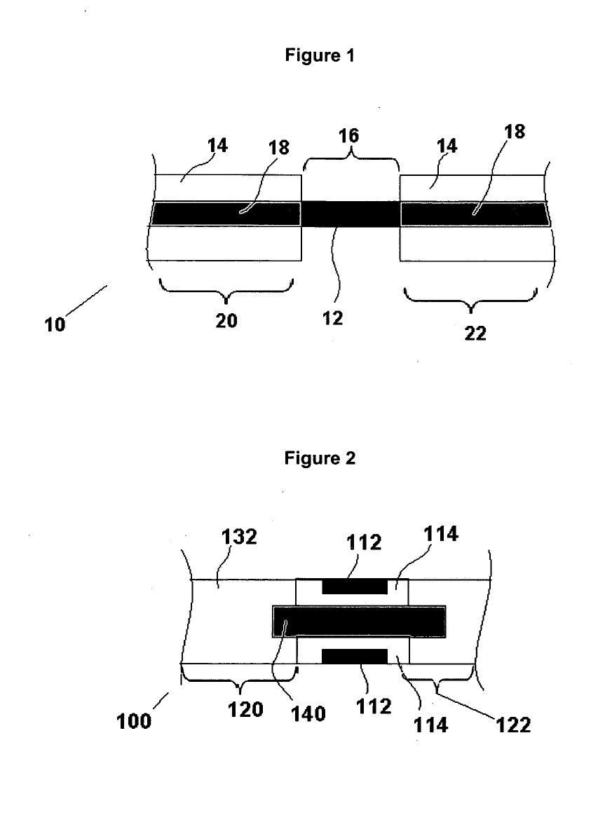

FIG. 1 shows a longitudinal cross sectional view of a portion of an implantable frame 10 segment comprising an removable material 12. The removable material 12 is severable or dissolvable by electrolysis in an electrolytic medium, such as an aqueous environment, within the human or mammalian body. The electrical current can be applied directly by placing an electrode in contact with the exposed region 16, or indirectly by introducing electrical current through the conductive frame portion 18. The removable material 12 is shown bridging a proximal frame portion 22 and a distal frame portion 20, and comprising an exposed region 16. The proximal frame portion 22 and the distal frame portion 20 each comprise an electrically conductive frame portion 18 core in electrically conducting contact with the removable material 12 and surrounded by an outer electrically insulating frame portion 14 that forms an outer surface of the implantable frame 10.

The electrode, conductive frame portion 18 and the frame 10 segment can have any suitable configuration that permits a desired rate and location of electrolytic removal of the removable material 12. For example, an electrode can induce a current in the conducting frame portion 18 by emitting an magnetic field. In other embodiments, the electrically insulating frame portion 14 can be omitted from the frame 10 segment. The composition or shape of the exposed region 16 or other portions of the conductive frame portion 18 can be altered to enhance, promote or direct the rate or intensity of the electrolytic process in selected areas. For example, the exposed region 16 can have a reduced cross sectional area compared to the conductive frame portion 18, for example to enhance or direct the electrolysis process in the exposed region 16. The cross sectional area of one or more portions of the removable material 12 can vary along the frame 10. For example, the removable material 12 at either end of an exposed region 16 can include areas of reduced cross sectional area, such as notches, grooves or fractures, positioned and configured to enhance or localize the electrolytic process within the exposed region 16. Preferably, a notch or taper is placed on opposite end of an exposed region 16 to promote electrolytic dissolution of the removable portion, for example to promote electrolytic dissolution by fracture propogation. A notch, fracture or groove can be have any suitable shape, such as a straight line, a “V” or a curved shape such as a “C” or “S” shape.

Returning to the embodiment illustrated in FIG. 1, the removable material 12 can be formed from materials that can be dissolved upon application of a sufficient electrical current by electrolysis in an ionic solution, such as blood or most other bodily fluids and including aqueous environments. The removable material 12 forms a portion of an implanted frame that is dissolvable or removable by electrolysis within the body. The removable material 12 can be electrolytically disintegrated by the application of a current in a manner providing for disintegration of the removable material 12 in a safe and predictable manner. The removable material 12 is preferably more susceptible to disintegration by electrolysis than the electrically conductive frame portion 18. More specifically, the removable material 12 is lower in the electromotive series than the material making up the conductive frame portion 18. For example the conductive frame portion 18 can be made of a material such as platinum or other noble metals, and the removable material 12 can be made of steel, stainless steel, nickel, nickel-titanium alloys, or other materials which will electrolytically dissolve in an aqueous fluid medium such as blood, saline solution, or other bodily fluid prior to the dissolution of the conductive frame portion 18.

The shape and size of the removable material 12 can vary depending on various design considerations and the intended use of the medical device. The removable material 12 can be configured to include an exposed region 16, where the electrolytic dissolution of the removable material 12 occurs. The length of the exposed region 16 is preferably approximately equal to its diameter to reduce the likelihood of multiple electrolytic etch sites on the exposed region 16. Preferably, the exposed region 16 has a length that is less than 0.50 inch, more preferably as shown as about 0.01 inch, and most preferably not longer than about 0.15 inch. The removable material 12 can have any suitable configuration and can be tapered or otherwise modified. Optionally, the exposed region 16 can be formed by coating the removable material 12 with an insulative polymer and removing a portion of the insulating polymer, such as described in U.S. Pat. No. 5,624,449 to Pham et al., to limit the area of removable material 12 to a more discrete region or point.

The removable material 12 can be optionally coated with any material that sufficiently separates or isolates the removable material 12 from a surrounding ionic solution. In some embodiments, the removable material 12 is coated with a bioabsorbable polymer, such as poly(lactic acid) (PLA). Other examples of other bioabsorbable polymers include: a polyester, a polyester-ethers, copoly(ether-esters), a poly(hydroxy acid), a poly(lactide), a poly(glycolide), or co-polymers and mixtures thereof. In another aspect, the bioabsorbable material is poly(p-dioxanone), poly(epsilon-caprolactone), poly(dimethyl glycolic acid), poly(D,L-lactic acid), L-polylactic acid, or glycolic acid, poly(lactide-co-glycolide), poly(hydroxybutyrate-co-valerate), poly(glycolic acid-co-trimethylene carbonate), poly(epsilon-caprolactone-co-p-dioxanone), poly-L-glutamic acid or poly-L-lysine, poly(hydroxy butyrate), polydioxanone, PEO/PLA or a co-polymer or mixture thereof. Bioabsorbable materials further include modified polysaccharides (such as cellulose, chitin, and dextran), modified proteins (such as fibrin and casein), fibrinogen, starch, collagen and hyaluronic acid. In general, these materials biodegrade in vivo in a matter of weeks or months, although some more crystalline forms can biodegrade more slowly.

In other embodiments, portions of the removable material 12 are coated with a biostable polymer such as parylene (polyxylylene). A portion of a parylene coating can be subsequently removed from the surface of the removable material 12 using a UV laser (excimer type) to cut a groove of about 1-3 mil in width to form a small exposed region 16. The exposed portion 16 of the removable material 12 is dissolved during the electrolysis process. The insulating frame portion 14 prevents or lessens current flow to the body vessel from the conductive frame portion 18 and/or concentrates the current flow through the removable material 12. Preferably, as shown in FIG. 1, insulating frame portion 14 surrounds removable material 12. The insulating frame portion 14 can have any suitable configuration, including a monolithic layer of a single polymer or thermoplastic, multiple layers of various polymers or thermoplastics, or an electrically insulative metallic oxide (alone or in combination with any number of polymers or thermoplastics).

Preferably, insulating frame portion 14 is comprised of a biocompatible, electrically insulative material such as polyfluorocarbons (e.g. TEFLON), polyethylene terephthalate (PET), polypropylene, polyurethane, polyimides, polyvinylchloride, silicone polymers, parylene, or combinations thereof. Parylene refers to a variety of polymers (e.g., polyxylylene) based on para-xylylene. These polymers are typically placed onto a substrate by vapor phase polymerization of the monomer. Parylene N coatings are produced by vaporization of a di(P-xylylene) dimer, pyrolization, and condensation of the vapor to produce a polymer that is maintained at a comparatively lower temperature. In addition to parylene-N, parylene-C is derived from di(monochloro-P-xylylene) and Parylene-D is derived from di(dichloro-P-xylylene). There are a variety of known ways to apply parylene to substrates. Their use in surgical devices has been shown, for instance, in U.S. Pat. No. 5,380,320 (Morris), U.S. Pat. No. 5,174,295 (Christian et al.), U.S. Pat. No. 5,067,491 (Taylor et al.), and the like. Alternatively, thermoplastic materials such as those disclosed in U.S. Pat. No. 5,944,733 to Engelson, the entirety of which is hereby incorporated herein by reference, are contemplated for use as adhesives in comprising the insulating frame portion 14, alone or in combination with the other polymers herein described. The thermoplastic, polymer or combination of such used to comprise insulating frame portion 14 may be formed in any number of ways. One technique, for example, is dipping or coating the conductive frame portion 18 and/or the removable material 12 in a molten or substantially softened polymer material, but other techniques as known in the art, such as shrink-wrapping, line-of-sight deposition in the form of a suspension or latex, or others may be used as well.

Biocompatible biostable polymers can optionally be used to form one or more coating layers on portions of the frame 10, including: poly(n-butyl-acrylate), poly(n-butyl methacrylate), poly 2-ethylhexyl acrylate, poly lauryl-acrylate, poly 2-hydroxy-propyl acrylate, polyvinyl chloride, polyvinyl methyl ether, polyvinylidene fluoride, polyvinylidene chloride, polyacrylonitrile, polystyrene, polyvinyl acetate, ethylenemethyl methacrylate copolymers, acrylonitrile-styrene copolymers, ethylene glycol diacrylate, ethylene glycol dimethacrylate, trimethylopropane triacrylate, trimethylopropane trimethacrylate, pentaerythritol tetraacrylate or pentaerythritol tetramethacrylate, 1,6-hexanediol dimethacrylate, diethyleneglycol dimethacrylate, N-methylol methacrylamide butyl ether, N-vinyl pyrrolidone, vinyl oleate, polyvinyl chloride, polyvinylidene fluoride, ABS resins, Nylon 66, rayon, rayon-triacetate, cellulose, cellulose acetate, cellulose butyrate, cellulose acetate butyrate, cellophane, cellulose nitrate, cellulose propionate, carboxymethyl cellulose, and polymers, copolymers or mixtures thereof.

Biocompatible and electrically resistive metallic oxide can also be used to form the insulating frame portion 14, alone or in combination with one or more thermoplastic or polymer layer. Oxides with a high dielectric constant, such as those of tantalum or titanium or their alloys, are preferred, with the various oxides of tantalum as most preferred. Such oxides can be formed in any number of ways. For example, they may be in the form of a deposited film, such as that made by plasma deposition of the base metal (e.g., in elemental or alloy form), or they may exist in the form of a sleeve or hypotube of the base metal that is welded, brazed, soldered, mechanically joined, or otherwise fixed to the conductive frame portion 18 and/or the removable material 12. This base metal layer can be subsequently oxidized (by imposition of the appropriate electrical current or other such excitation, such as by welding during assembly of the device) to form the desired electrically insulative oxide layer. Alternatively, the oxide may be deposited directly in oxide form by any number of techniques that does not require subsequent oxidation of the base metal in elemental or alloy form. Noble metal coatings, such as gold, plated or otherwise placed on a device can also be used as a insulating frame portion 14.

The insulating frame portion 14 can have any suitable thickness (as measured radially outward from center of the frame segment) that provides an adequate level of electrical insulation for a desired use. For example, the thickness can range from 0.002 inch to 0.040 inch, with 0.001 inch to 0.018 inch being preferred and 0.003 inch to 0.0010 inch as most preferred. The optimal thickness of each layer will depend on the desired thermal, electrical and mechanical properties of the insulating frame portion 14, the types and combinations of materials used, dimensional constraints relative to the removable material 12 and the conductive frame portion 18, as well as manufacturing, engineering, cost and other factors. For instance, the thickness of the insulating frame portion 14 can range from one or a few hundred angstroms if an oxide layer was used, or thicker if a polymer or thermoplastic was used.

The conductive frame portion 18 can provide an electrical pathway between a current source and one or more removable material 12 portions of an implantable frame, to transmit electrical current readily therebetween. Alternatively, the conductive frame portion 18 can also provide an electrical pathway between two or more regions of the implantable frame comprising removable material 12. A conductive frame portion 18 can be made from any biocompatible, electrically conductive material. While conducting electrical current, the conductive frame portion 18 preferably does not decompose prior to the dissolution of an adjacent removable material 12. Preferably, the conductive frame portion 18 is formed from a suitable metal such as platinum, stainless steel hypotubing or a superelastic material such as nitinol. The conductive frame portion 18 may be connected to the removable material 12 by any suitable method, including welding, brazing, soldering, mechanically joining (as by crimping, for example) or otherwise connecting.

FIG. 2 shows a longitudinal cross sectional view of a portion of a second implantable frame 100 segment comprising a removable material 112 configured as a rigid annular ring positioned the outside surface of the implantable frame 100. The removable material 112 ring is fitted in a groove of a ring of insulating material 114 that electrically insulates the removable material 112 from the remainder of the implantable frame segment 100. The implantable frame segment also includes a flexible core member 140 providing a durable and flexible connection between a proximal frame portion 122 and a distal frame portion 120. The flexible core member 140 is formed from any material with a desired level of durability and flexibility, and is preferably formed from a thermoformable polymer or rubber. The insulating material 114 can be formed from one or more of the materials discussed above with reference to the insulating frame portion 14. The removable material 112 can be selected from one or more materials discussed above with reference to the removable material 12.

A flexible joint can be introduced between the proximal frame portion 122 and the distal frame portion 120 by electrolytically dissolving the removable material 112 ring. With the removable material 112 in place, the proximal frame portion 122 and the distal frame portion 120 are fixed with respect to one another. The removable material 112 can be dissolved by applying an electrical current, rendering the proximal frame portion 122 and the distal frame portion 120 moveable with respect to one another by bending the flexible core member 140.

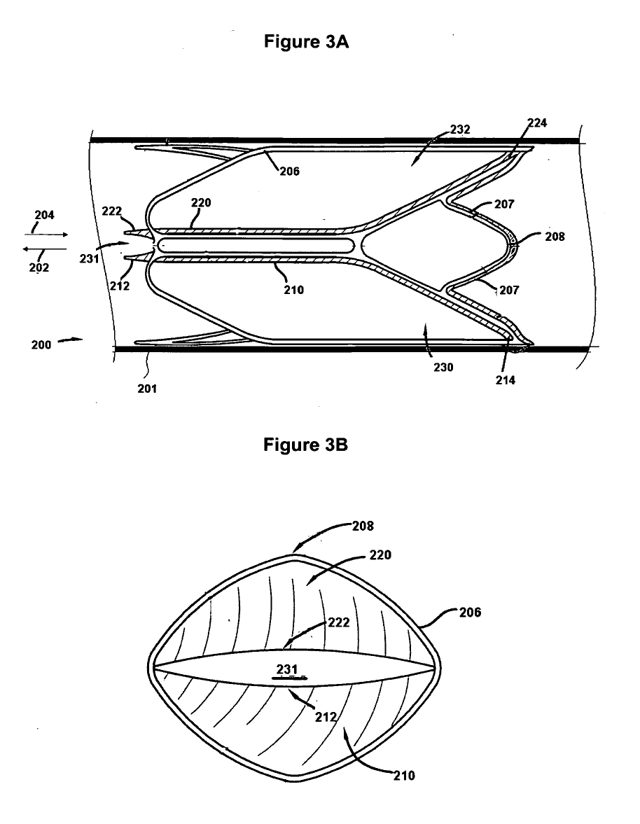

Referring to FIG. 3A, an implantable valve 200 is shown within a body vessel segment 201. The implantable valve 200 includes a support frame 206 with a removable portion 208 as part of a bridging member 207 that exerts a radially outward force. The implantable valve is configured to permit fluid to flow in substantially a first direction 202. Removal of the removable portion 208 of the bridging member 207 lowers the radial stiffness of the implantable valve within the body vessel segment 201. The support frame 206 defines a substantially cylindrical interior lumen containing a pair of valve leaflets 210 and 220. The support frame 206 also includes unattached frame portions defining a portion of the substantially cylindrical interior lumen, without being attached to the leaflets 210 and 220.

The implantable valve 200 can be formed by attaching two substantially similar pliable valve leaflets 210 and 220 to a support frame 206. Preferably, (n−1) edges of each leaflets 210 and 220 are attached to the support frame 206, where (n) is the total number of sides. The leaflets 210 and 220 are preferably substantially similar or identical and are positioned in an opposable configuration. Although two leaflets are shown in the implantable valve 200, valves can have any number of leaflets, including 1, 2, 3, 4, 5, 6, 7, 8, or more leaflets. One edge of each of the leaflets 210 and 220 form leaflet free edges 212 and 222, respectively, that are opposably positioned to cooperably define a valve orifice. The leaflets 210 and 220 are configured in any shape and formed of any biocompatible material to provide leaflet free edges 212 and 222 to open or close the valve orifice in response to changes in direction of fluid flow within the body vessel segment 201. Fluid flowing in a first direction 202 forces the body of the leaflets 210 and 220 apart from each other, thereby parting the leaflet free edges 212 and 222, permitting fluid to flow through the valve in the first direction 202.

Each leaflet can comprise one or more body vessel contact edges, such as edges 214 and 224, that are attached to the frame and contact the inner wall of a body vessel, forming open sinus regions 230 and 232 bounded by the inner wall of the body vessel segment 201 on the opposite sides and by the leaflets 210 and 220. When fluid flows in the retrograde direction 204, the fluid collects in the sinus regions 230 and 232. Fluid collecting in the sinus regions 230 and 232 exerts pressure radially inward, urging the leaflets 210 and 220 toward each other and resulting in closure of the valve orifice 231 as the leaflet free edges 212 and 222 contact each other.

The implantable frame 206 is preferably formed from a self-expanding material configured to exert a radial outward force securing the implantable frame 206 against the body vessel segment 201. Alternatively, the implantable frame can be formed from material that is not typically self-expanding, such as stainless steel or a cobalt chromium alloy, that is balloon expanded and secured in the body vessel segment 201 by other means. For example, small barbs can be positioned on the surface of the implantable frame 206 to engage the wall of the body vessel segment 201. Removal of the removable portion 208 can decrease the radial strength of the implantable frame 206. The bridging member 207 can optionally exert an outward radial bias separating the leaflets 210 and 220 that is reduced or eliminated upon removal of the removable portion 208.

Endovascular Electrolysis of Removable Region

Frames comprising a removable region can be placed in a body vessel and later altered or removed by the electrolytic dissolution or reduction of the removable region. The electrolytic dissolution of the removable region can be performed within a body vessel using an electrically conducting member such as a catheter having an electrode, or by inducing an electrical current by external application of an electromagnetic field.

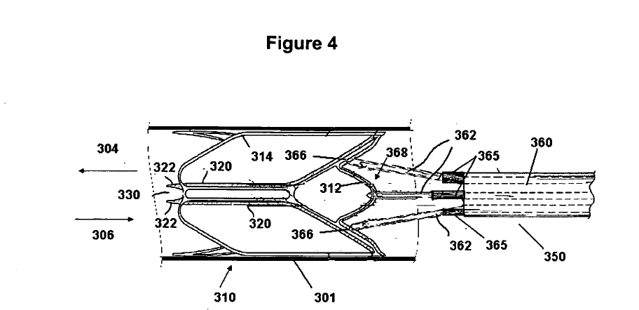

An electrically conducting member can have any suitable configuration, including one or more extendable electrodes or an annular distal electrode, that permits electrical conducting contact between the electrode and one or more removable region of an implanted frame. FIG. 4 shows a first intraluminal conducting member exemplified as a catheter 350 deployed in electrically conducting contact with an implanted valve 310 within a body vessel segment 301. The implanted valve 310 comprises a support frame 314 having multiple removable portions 312 and a pair of flexible leaflets 320 attached to the support frame 314. The pair of leaflets 320 includes a pair of opposably positioned flexible free edges 322 that cooperatively define a valve orifice 330. As described above with respect to the implantable valve 200, the valve orifice 330 opens to permit fluid to flow in a first direction 304 and closes to substantially prevent fluid flow in the opposite retrograde direction 306. The removable portions 312 are positioned at certain bends of the support frame 314. The removable portions 312 can have any configuration, including the configurations illustrated in FIG. 1 to permit breaking of the frame upon dissolution of the removable portions 312, or the configuration illustrated in FIG. 2 to permit bending of the frame upon dissolution of the removable portion 312.

The catheter 350 includes three electrode members 362 that can be deployed in a radially outward manner. The electrode members 362 can have a radially compressed configuration and various radially expanded configurations. Preferably, the electrode members 362 are designed and configured to self-expand in a radially outward direction, as shown in FIG. 4, in the absence of the application of radial compression. For example, the electrode members 362 can comprise a self-expanding material such as a superelastic nickel-titanium alloy, or can be spring biased in a radially outward fashion. The electrode members 362 can be formed from any electrically conducting material 366, including the materials described above for the conductive frame portion 18 in FIG. 1. The electrically conducting material 366 may comprise any conductive biocompatible material. For example, electrically conducting material 366 may comprise conductive metals and their alloys (for example, steel, titanium, copper, platinum, nitinol, gold, silver or alloys thereof), carbon (fibers or brushes), electrically conductive doped polymers or epoxies, or any combination thereof. The electrode member 362 can have any suitable configuration permitting electrically conducting contact between at least a portion of the electrode and implanted frame.

The catheter 350 also includes a sheath member 360 with a laminate tubular structure that is longitudinally translatable relative to the pair of electrode members 362. Translation of the sheath member 360 toward a radiopaque catheter tip 368 compresses the electrode members 362 radially inward toward the radially compressed configuration. A portion of the electrode members 362 are preferably enclosed by an insulating material 365 that can be formed from any material suitable for the insulating frame portion 14 above. One function of the insulating material 365 is to protect the body vessel from undesirable contact with the conducting portions of the electrode members 362. Another possible function of the insulating material 365 is to provide a housing to mechanically guide a conducting portion of the electrode members 362 to connect with a removable region 312 of an implanted frame 314. Other catheter constructions may be used without departing from the scope of the invention.

In operation, the catheter 350 is inserted in a body vessel with the electrode members 362 in the radially compressed configuration and covered by the sheath member 360. Conventional catheter insertion and navigational techniques may be used to access the implanted valve 310. To position the distal end of catheter 350 at the site, often by locating its distal end through the use of radiopaque marker material and fluoroscopy, the catheter 350 is translated to the implantable frame 310. The distal portion of the catheter 350 is positioned proximate to the implantable frame 310, for example by radiographic monitoring of the position of the catheter tip 368. The sheath member 360 is then translated away from the catheter tip 368 relative to the electrode members 362, thereby deploying the electrode members 362 in a radially outward direction and contacting the electrically conducting material 366 with the implanted frame.

Electrical current is passed from the electrically conducting material 366 through on or more of the removable regions 312 until the removable regions 312 are electrolytically disintegrated. A positive electric current of approximately 0.01 to 2 milliamps at 0.1 to 6 volts is next applied to electrically conducting material 366 by any suitable power supply (not shown). For example, in one embodiment, a voltage of 3.0V at 1.0 milliamp can dissolve a 0.005 inch long, 0.003 inch diameter removable frame portion in a period of between about 44-97 seconds. Typically, the negative pole of power supply is placed in electrical contact with the skin. Preferably, the current can be allowed to return through a conducting region of the catheter or a guidewire. The removable regions 312 are typically completely dissolved or eroded by electrolytic action, typically between 5 seconds to 10 minutes. Upon dissolution of the removable regions 312, the implantable frame 314 can have a decreased radial flexibility, can exert less mechanical tension on the leaflet 320 material, and/or can have flexible bends in regions that were previously rigid. The electrode members 362 are then returned to the radially compressed state by translation of the sheath member 360 toward the catheter tip 368, and the catheter 350 is removed from the body vessel. Preferably, the implantable frame 314 includes an electrically conducting material between removable regions 312 configured to conduct electrical current applied to any portion of the implantable frame 314, including a first removable region: 312, to other removable regions 312 that can also be dissolved or removed simultaneously and without separately contacting an electrode member 362 to each individual removable region 312. The catheter 350, or any portion thereof, may take any number of forms that effectively transmit electric current to removable region 312.

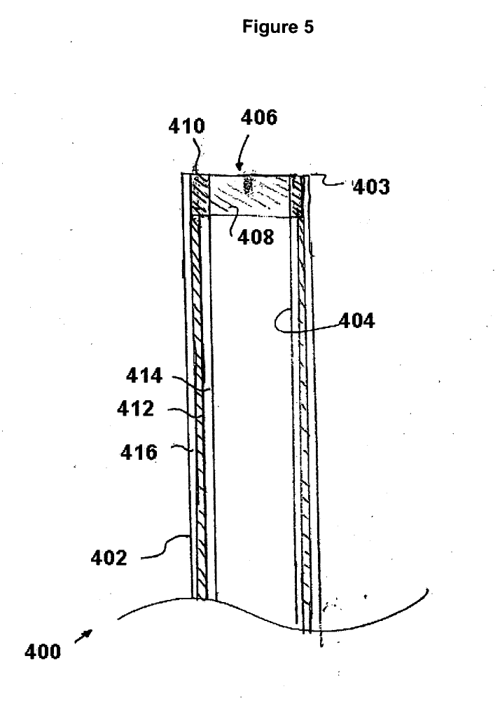

FIG. 5 shows a second intraluminal conducting member configured as a catheter 402 having a distal electrode 410 in communication with a current source (not shown) through an annular conducting region 412 running through the body of the catheter 402. The annular conducting region 412 is enclosed in an inner insulating tube 414 and an outer insulating tube 416. The inner insulating tube 414 defines an interior lumen 404. The inner insulating tube 414 and the outer insulating tube 416 can be formed from any biocompatible material, preferably a thermoformable polymer that provides adequate levels of flexibility and durability. Alternatively, annular conducting region 412 can be in the form of a wire or ribbon whose distal end is coupled, for example by welding, to the distal electrode 410. Annular conducting region 412 extends from distal electrode 410 between inner and outer insulating tube members 414 and 416 to proximal end portion of catheter 402 where it can be electrically connected to a power supply either directly or with a lead as would be apparent to one of ordinary skill in the art.

The inner and outer insulating tube members 414 and 416 prevent current flow from the conducting region 412 into the surrounding ionic medium (such as blood) surrounding the catheter. The inner and outer insulating tube members 414 and 416 may be comprised of an electrically insulative polymer or polymers as described above, and may additionally or singly comprise an electrically insulative metallic oxide such as tantalum oxide or the like. The insulating tube members 414 and 416 can be formed within the body vessel upon application of an electrical current to the conducting region 412. For example, the conducting region 412 can comprise an oxide forming material which, under the imposition of an electrical current, will form an oxide skin, particularly in an ionic medium such as saline solution, blood or other bodily fluids. One such material is the metal tantalum and certain alloys comprising tantalum, that form a tantalum oxide coating that can serve as the insulating tube members 414 and 416. Other insulation-forming materials or oxide forming materials can be used in the conducting region 412 such as zirconiums, its alloys and related materials, which form or may be made to form exterior resistive layers by various techniques including nitriding or the like that can be performed in situ. The conducting region 412 can also comprise platinum, stainless steel, gold or other materials. The inner insulating tube members 414 may, for example, be a metallic braid, distal electrode ring 410 may, for example, be a platinum or platinum alloy hypotube. The annular conducting region 412 and distal electrode 410 can have any suitable configuration that permits a desired amount of contact between the electrode and a portion of an implanted frame, including a distal electrode shaped as a ring connected to an annular conducting region 412. The distal electrode 310 may also extend beyond the distal end of catheter 402 to ensure electrical contact with a portion of an implanted frame within a body vessel. For example, the distal electrode 410 can have other configurations, such as a tubular braided structure such as described in U.S. Pat. No. 6,059,779. A braided electrode configuration can have the advantage of allowing the designer to vary the stiffness of the catheter by varying the mesh size of the braid along the length of the catheter. Although FIG. 5 shows distal electrode 410 to be substantially flush with the distal end 403 of catheter 402, the distal electrode 410 can be spaced inwardly from the distal end 403 of catheter 402 to eliminate or minimize interference with other vaso-occlusive members. Likewise, the distal electrode 410 can be spaced radially outward from the distal surface of catheter 402 to ensure conductive contact with an implanted frame.

The distal electrode 410 can be formed continuously with the conducting region 412, for example as one continuous tube of conducting material. Alternatively, the distal electrode 410 can be joined to the conducting region 412 in any manner that permits conduction of electrical current between the two, including welding, brazing, soldering, gluing, or otherwise electrically and fixedly attaching.

Implantable Frames

An implantable frame is preferably of a size sufficiently small to be advanced through a catheter (not shown) that is appropriately sized for accessing the targeted vascular site. The frame is preferably an expandable frame having a compressed configuration small enough to be implanted from delivery catheter within a body vessel. Suitable support frames can have a variety of configurations, including braided strands, helically wound strands, ring members, consecutively attached ring members, tube members, and frames cut from solid tubes. Also, suitable frames can have a variety of sizes. The exact configuration and size chosen will depend on several factors, including the desired delivery technique, the nature of the vessel in which the device will be implanted, and the size of the vessel. A frame structure and configuration can be chosen to facilitate maintenance of the device in the vessel following implantation.

The frame can, in one embodiment, comprise a plurality of struts. Struts are structures that can resist compression along the longitudinal axis of the strut. Struts can be an identifiable segment of an elongated frame member, for example separated by bends in the member, individual segments joined together, or any combination thereof. Struts can have any suitable structure or orientation to allow the frame to provide desirable radial strength properties to the frame. For example, struts can be oriented substantially parallel to, substantially perpendicular to, or diagonal to the longitudinal axis of a tubular frame, or some combination thereof. Struts can be straight or arcuate in shape, and can be joined by any suitable method, or can form one or more distinct rings.

In one aspect, implantable frames comprise a serpentine (or zig-zag) plurality of struts having substantially equal lengths joined together in a reversing pattern. In another aspect, implantable frames comprise repeating S-shaped hinge regions or repeating Z-shaped hinge regions. The latter pattern is commonly referred to a zig-zag stent.

Various constructs of the elongate elements, fibers and threads can be formed utilizing well known techniques, e.g., braiding, plying, knitting, weaving, that are applied to processing natural fibers, e.g., cotton, silk, etc., and synthetic fibers made from synthetic bioabsorbable polymers (including poly(glycolide), poly(lactic acid), poly(caprolactone) and copolymers thereof, nylon, cellulose acetate, and the like. See, e.g., Mohamed, American Scientist, 78: 530-541 (1990). Specifically, collagen thread is wound onto cylindrical stainless steel spools. The spools are then mounted onto the braiding carousel, and the collagen thread is then assembled in accordance with the instructions provided with the braiding machine. In one particular run, a braid was formed of four collagen threads, which consisted of two threads of uncrosslinked collagen and two threads of crosslinked collagen.

The dimensions of the implantable frame will depend on its intended use. Typically, the implantable frame will have a length in the range from 0.5 cm to 10 cm, usually being from about 1 cm to 5 cm, for vascular applications. The small (radially collapsed) diameter of a cylindrical frame will usually be in the range from about 1 mm to 10 mm, more usually being in the range from 1.5 mm to 6 mm for vascular applications. The expanded diameter will usually be in the range from about 2 mm to 30 mm, preferably being in the range from about 2.5 mm to 15 mm for vascular applications. The body segments may be formed from conventional malleable materials used for body lumen stents and grafts, typically being formed from metals.

The radial strength of a frame is preferably measured using a Radial Force Gauge. One preferred Radial Force Gauge the RX600 Radial Expansion Force Gage equipment from Machine Solutions Inc. (MSI). A Radial Force Gauge measures the radial strength of both balloon expandable and self-expanding stent and stent graft products during expansion and compression. The RX600 equipment uses a segmental compression mechanism controlled by a micro-stepping linear actuator that is designed to provide an extremely low friction testing environment. Preferably, the Radial Force Gauge maintains resolution at force levels from 0 to 80 Newtons, for example using a software-controlled interchangeable linear force transducer, or other suitable means. The Radial Force Gauge preferably measures the hoop strength of the frame. Optionally, the Radial Force Gauge allows the hoop strength of the frame to be visualized and recorded as the product is cycled through programmed open and close diameters.

Remodelable Materials

A medical device can comprise a support frame and a remodelable material attached to the frame, such as a valve leaflet formed from a remodelable material. Preferably, the remodelable material is subject to a mechanical load adequate to allow remodeling of the remodelable material when the frame a first radial strength, prior to electrolytic dissolution of a removable portion of the frame, and a reduced mechanical load after electrolytic dissolution of the removable portion.

Mechanical loading of remodelable material during the remodeling process can advantageously influence the remodeling process. For example, the remodeling process of one type of remodelable material, extracellular matrix (ECM), is more effective when the material is subject to certain types and ranges of mechanical loading during the remodeling process. See, e.g., M. Chiquet, “Regulation of extracellular matrix gene expression by pressure,” Matrix Biol. 18(5), 417-426 (October 1999). Applying mechanical forces to a remodelable material during the remodeling process is believed to affect processes such as signal transduction, gene expression and contact guidance of cells. Various references describe the influence of mechanical loading on remodelable materials, such as extracellular matrix material (ECM). For example, mediation of numerous physiological and pathological processes by vascular endothelial cells is influenced by mechanical stress, as discussed, for example, in Chien, Shu et al., “Effects of Mechanical Forces on Signal Transduction and Gene Expression in Endothelial Cells,” Hypertension 31(2): 162-169 (1998). Expression of bioactive agents can be stimulated by mechanical stress on certain cells involved in remodeling processes, such as fibroblasts, as discussed, for example, by Schild, Christof et al., “Mechanical Stress is Required for High-Level Expression of Connective Tissue Growth Factor,” Experimental Cell Research, 274: 83-91 (2002). Furthermore, another study suggests that fibroblasts attached to a remodelable material such as a strained collagen matrix produce increased amounts of ECM glycoproteins like tenascin and collagen XII compared to cells in a relaxed matrix. Chiquet, Matthias, et al., “Regulation of Extracellular Matrix Synthesis by Mechanical Stress,” Biochem. Cell. Biol., 74:737-744 (1996). Other studies of remodelable material have found that remodeling processes are sensitive to alterations in mechanical load. See, e.g., Wong, Mary et al., “Cyclic Compression of Articular Cartilage Explants is Associated with Progressive Consolidation and Altered Expression Pattern of Extracellular Matrix Proteins,” Matrix Biology, 18: 391-399 (1999); Grodzinsky, Alan J. et al., “Cartilage Tissue Remodeling in Response to Mechanical Forces,” Annual Review of Biomedical Engineering, 2: 691-713 (2000). In addition, the alignment of cells with respect to mechanical loads can affect remodeling processes, as studied, for example, by V C Mudera et al., “Molecular responses of human dermal fibroblasts to dual cues: contact guidance and mechanical load,” Cell Motil. Cytoskeleton, 45(1):1-9 (June 2000). These references are incorporated herein by reference.

In some embodiments, upon implantation in a body vessel, a remodelable material can be subject to both a mechanical load, for example from the manner of attachment to a frame, as well as a variable shear stress from the fluid flow within the body vessel. For example, Helmlinger, G. et al., disclose a model for laminar flow over vascular endothelial cells in “Calcium responses of endothelial cell monolayers subjected to pulsatile and steady laminar flow differ,” Am. J. Physiol. Cell Physiol. 269:C367-C375 (1995).

Shear forces within a body vessel can also influence biological processes involved in remodeling. For example, the role of hemodynamic forces in gene expression in vascular endothelial cells is discussed by Li, Y. S. et al., “The Ras-JNK pathway is involved in shear-induced gene expression,” Mol. Cell. Biol., 16(11): 5947-54 (1996). Many other studies of the range of shear forces and the effect of shear forces on the remodeling process are found in the art. Using these references and others, one skilled in the art can select a level of mechanical loading that, when taking into account the range of fluid flow shear forces within a body vessel, will provide optimal mechanical loading conditions for remodeling of the remodelable material.

Any suitable remodelable material may be used. Preferably, the remodelable material is an extracellular matrix material (ECM), such as small intestine submucosa (SIS). To facilitate ingrowth of host or other cells during the remodeling process, either before or after implantation, a variety of biological response modifiers may be incorporated into the remodelable material. Appropriate biological response modifiers may include, for example, cell adhesion molecules, cytokines, including growth factors, and differentiation factors. Mammalian cells, including those cell types useful or necessary for populating the resorbable stent of the present invention, are anchorage-dependent. That is, such cells require a substrate on which to migrate, proliferate and differentiate.

A remodelable material, can undergo biological processes such as angiogenesis when placed in communication with a living tissue, such that the remodelable material is biologically transformed into material that is substantially similar to said living tissue in cellular composition. Unless otherwise specified herein, a “remodelable material” can include a single layer material, or multiple layers of one or more materials that together undergo remodeling when placed in communication with living tissue. Preferably, a remodelable material undergoes a desired degree of remodeling upon contact for about 90 days or less with living tissue of the type present at an intended site of implantation, such as the interior of a body vessel. One example of a remodeling process is the migration of cells into the remodelable material. Migration of cells into the remodelable material can occur in various ways, including physical contact with living tissue, or recruitment of cells from tissue at a remote location that are carried in a fluid flow to the remodelable material. In some embodiments, the remodelable material can provide an acellular scaffold or matrix that can be populated by cells. The migration of cells into the remodelable material can impart new structure and function to the remodelable material. In some embodiments, the remodelable material itself can be absorbed by biological processes. In some embodiments, fully remodeled material can be transformed into the living tissue it is in contact with through cellular migration from the tissue into the remodelable material, or provide the structural framework for tissue. Non-limiting examples of remodelable materials, their preparation and use are also discussed herein.

Any remodelable material, or combination of remodelable materials can be used as a remodelable material for practicing the present invention. For instance, naturally derived or synthetic collagen can provide retractable remodelable materials. Naturally derived or synthetic collagenous material, such as extracellular matrix material, are suitable remodelable materials. Examples of remodelable materials include, for instance, submucosa, renal capsule membrane, dura mater, pericardium, serosa, and peritoneum or basement membrane materials. Collagen can be extracted from various structural tissues as is known in the art and reformed into sheets or tubes, or other shapes. The remodelable material may also be made of Type III or Type IV collagens or combinations thereof. U.S. Pat. Nos. 4,950,483, 5,110,064 and 5,024,841 relate to such remodelable collagen materials and are incorporated herein by reference. Further examples of materials useful as remodelable materials include: compositions comprising collagen matrix material, compositions comprising epithelial basement membranes as described in U.S. Pat. No. 6,579,538 to Spievack, the enzymatically digested submucosal gel matrix composition of U.S. Pat. No. 6,444,229 to Voytik-Harbin et al., materials comprising the carboxy-terminated polyester ionomers described in U.S. Pat. No. 5,668,288 to Storey et al., collagen-based matrix structure described in U.S. Pat. No. 6,334,872 to Termin et al., and combinations thereof. In some embodiments, submucosal tissues for use as remodelable materials include intestinal submucosa, stomach submucosa, urinary bladder submucosa, and uterine submucosa. A specific example of a suitable remodelable material is intestinal submucosal tissue, and more particularly intestinal submucosa delaminated from both the tunica muscularis and at least the tunica mucosa of warm-blooded vertebrate intestine.

One preferred type of remodelable material is extracellular matrix material derived from submocosal tissue, called small intestine submucosa (SIS). Additional information as to submucosa materials useful as ECM materials herein can be found in U.S. Pat. Nos. 4,902,508; 5,554,389; 5,993,844; 6,206,931; 6,099,567; and 6,375,989, as well as published U.S. Patent Applications US2004/0180042A1 and US2004/0137042A1, which are all incorporated herein by reference. For example, the mucosa can also be derived from vertebrate liver tissue as described in WIPO Publication, WO 98/25637, based on PCT application PCT/US97/22727; from gastric mucosa as described in WIPO Publication, WO 98/26291, based on PCT application PCT/US97/22729; from stomach mucosa as described in WIPO Publication, WO 98/25636, based on PCT application PCT/US97/23010; or from urinary bladder mucosa as described in U.S. Pat. No. 5,554,389; the disclosures of all are expressly incorporated herein.

The remodelable material can be isolated from biological tissue by a variety of methods. In general, a remodelable material such as an extracellular matrix (ECM) material can be obtained from a segment of intestine that is first subjected to abrasion using a longitudinal wiping motion to remove both the outer layers (particularly the tunica serosa and the tunica muscularis) and the inner layers (the luminal portions of the tunica mucosa). Typically the SIS is rinsed with saline and optionally stored in a hydrated or dehydrated state until use as described below. The resulting submucosa tissue typically has a thickness of about 100-200 micrometers, and may consist primarily (greater than 98%) of acellular, eosinophilic staining (H&E stain) ECM material.

Preferably, the source tissue for the remodelable material is disinfected prior to delamination by using the preparation disclosed in U.S. Pat. No. 6,206,931, filed Aug. 22, 1997 and issued Mar. 27, 2001 to Cook et al., and US Patent Application US2004/0180042A1 by Cook et al., filed Mar. 26, 2004, published Sep. 16, 2004 and incorporated herein by reference in its entirety. Most preferably, the tunica submucosa of porcine small intestine is processed in this manner to obtain the ECM material. This method is believed to substantially preserve the aseptic state of the tela submucosa layer, particularly if the delamination process occurs under sterile conditions. Specifically, disinfecting the tela submucosa source, followed by removal of a purified matrix including the tela submucosa, e.g. by delaminating the tela submucosa from the tunica muscularis and the tunica mucosa, minimizes the exposure of the tela submucosa to bacteria and other contaminants. In turn, this enables minimizing exposure of the isolated tela submucosa matrix to disinfectants or sterilants if desired, thus substantially preserving the inherent biochemistry of the tela submucosa and many of the tela submucosa's beneficial effects.

An alternative to the preferred method of ECM material isolation comprises rinsing the delaminated biological tissue in saline and soaking it in an antimicrobial agent, for example as disclosed in U.S. Pat. No. 4,956,178. While such techniques can optionally be practiced to isolate ECM material from submucosa, preferred processes avoid the use of antimicrobial agents and the like which may not only affect the biochemistry of the matrix but also can be unnecessarily introduced into the tissues of the patient. Other disclosures of methods for the isolation of ECM materials include the preparation of intestinal submucosa described in U.S. Pat. No. 4,902,508, the disclosure of which is incorporated herein by reference. Urinary bladder submucosa and its preparation is described in U.S. Pat. No. 5,554,389, the disclosure of which is incorporated herein by reference. Stomach submucosa has also been obtained and characterized using similar tissue processing techniques, for example as described in U.S. patent application Ser. No. 60/032,683 titled STOMACH SUBMUCOSA DERIVED TISSUE GRAFT, filed on Dec. 10, 1996, which is also incorporated herein by reference in its entirety.

Valve Members

A medical device can comprise a means for regulating fluid through a body vessel. In some embodiments, the fluid can flow through an implantable frame, while other embodiments provide for fluid flow through a lumen defined by the frame. In some aspects, a frame and a first valve member are connected to a frame.

A valve member, according to some aspects, can comprise a valve member, such as a leaflet comprising a free edge, responsive to the flow of fluid through the body vessel. A “free edge” refers to a portion of a leaflet that is not attached to a frame, but forms a portion of a valve orifice. Preferably a leaflet free edge is a portion of the edge of the leaflet that is free to move in response to the direction of fluid flow in contact with the leaflet, independently of the movement of the frame.

Preferably, one or more valve members attached to a frame can permit fluid to flow through a body vessel in a first direction while substantially preventing fluid flow in the opposite direction. A valve leaflet is one type of valve member. In some embodiments, the valve member comprises an extracellular matrix material, such as small intestine submucosa (SIS). The valve member can be made from any suitable material, including a remodelable material or a synthetic polymer material. Medical devices comprising a frame and a valve member can be used to regulate fluid flow in a vein, for example to treat venous valve incompetency. For example, one or more medical devices comprising a frame and one or more valve members can be implanted in a vein with incompetent venous valves so as to provide a valve to replace the incompetent valves therein.

A wide variety of materials acceptable for use as the valve members are known in the art, and any suitable material can be utilized. The material chosen need only be able to perform as described herein, and be biocompatible, or able to be made biocompatible. Examples of suitable materials include flexible materials, natural materials, and synthetic materials. Extracellular matrix (ECM) materials, such as submucosa or collagen, are one preferred examples of a suitable natural materials for a valve member. Small intestine submucosa (SIS) is particularly well-suited for use as a valve member, such as a leaflet. A valve member can comprise a suitable synthetic material including polymeric materials, such as polypropylene, expanded polytetrafluoroethylene (ePTFE), polyurethane (PU), polyethylene terphthalate (PET), silicone, latex, polyethylene, polypropylene, polycarbonate, nylon, polytetrafluoroethylene, polyimide, polyester, and mixture thereof, or other suitable materials.

A valve member can be attached to an implantable frame with any suitable attachment mechanism, such as sutures, adhesives, bonding, tissue welding, self-adhesion between regions of the material, chemical adhesion between the valve member material and the frame, cross-linking and the like. The attachment mechanism chosen will depend on the nature of the frame and valve members. Sutures provide an acceptable attachment mechanism when SIS or other ECM materials are used as the valve members with a metal or plastic frame.

The device can include any suitable number of valve members. The valve members need only be able to provide the functionality described herein. The specific number chosen will depend on several factors, including the type and configuration of the frame. Some aspects provide medical devices comprising 1, 3, 4, 5, 6, 7, 8 or more valve members. The valve members can be arranged in any suitable configuration with respect to one another and the frame. In one preferred embodiment, a medical device can comprise a frame and three valve members that are leaflets comprising free edges. In another preferred embodiment, a medical device can comprise one leaflet having a free edge that can sealably engage the interior of a vessel wall. Other suitable configurations of valve members are provided by further embodiments, including differently shaped valve members, and different points of attachment by valve members to the frame.

In some aspects, the frame provides one or more structural features that protect a valve member. For example, the frame can include a portion positioned between a portion of a leaflet and the interior wall of a body vessel upon implantation. Another example of a protecting feature in a frame includes arms or members of the frame extending between portions of a leaflet and the inner wall of a body vessel. As another example, a narrowed portion of an inner diameter of a frame around a leaflet can protect a portion of the leaflet from adhering to the inner wall of a body vessel upon implantation of a medical device therein. In one embodiment, the leaflet can comprise a remodelable material and the protecting structural feature of the frame can be bioabsorbed gradually in a time period sufficient for remodeling of at least a portion of the leaflet. Bioabsorption of the protecting feature of the frame can also gradually decrease the radial strength of the frame. In another embodiment, the protecting feature of the frame can fracture in a controlled manner, for instance by microfractures along a portion of the frame, after a suitable period of implantation (for example after about 30 days post implantation). Frames that comprise materials that decrease frame radial strength upon implantation by other means such as the absorption of fluid, responsive to changes in pH or body temperature, or various biochemical processes can also be used, for example as a structural feature to protect a leaflet or portion thereof from undesirable contact with the inner wall of a body vessel.

The overall configuration, cross-sectional area, and length of the valve support frame will depend on several factors, including the size and configuration of the device, the size and configuration of the vessel in which the device will be implanted, the extent of contact between the device and the walls of the vessel, and the amount of retrograde flow through the vessel that is desired.

In devices including multiple openings that permit a controlled amount of fluid flow in the second, opposite direction to flow through the vessel in which the device is implanted, the total open area of all openings can be optimized as described above, but it is not necessary that the individual openings have equivalent total open areas.

In one aspect, the method comprises the step of attaching a first valve member to a frame. The valve member can be responsive to the flow of fluid through the frame, and adapted to permit fluid flow through said vessel in a first direction or substantially prevent fluid flow through said vessel in a second, opposite direction. The frame can have a longitudinal axis, a first radial compressibility along a first radial direction that is less than a second radial compressibility along a second radial direction.

Implantable Frame Materials

Implantable frames can be constructed of any suitable material. Suitable materials are biocompatible. Preferably, the frame materials and design configurations are selected to reduce or minimize the likelihood of undesirable effects such as restenosis, corrosion, thrombosis, arrhythmias, allergic reactions, myocardial infarction, stroke, or bleeding complications. Examples of suitable materials include, without limitation: stainless steel, titanium, niobium, nickel titanium (NiTi) alloys (such as Nitinol) and other shape memory and/or superelastic materials, MP35N, gold, tantalum, platinum or platinum alloy including platinum iridium, Elgiloy, Phynox (a cobalt-based alloy), or any cobalt-chromium alloy. The stainless steel may be alloy-type: 316L SS, Special Chemistry per ASTM F138-92 or ASTM F139-92 grade 2, Special Chemistry of type 316L per ASTM F138-92 or ASTM F139-92 Stainless Steel for Surgical Implants.

In some embodiments, the frame is formed partially or completely of alloys such as nitinol, which is believed to consist essentially of 55% Ni, 45% Ti, and which have superelastic (SE) characteristics. When a frame is formed from superelastic nickel-titanium (NiTi) alloys, the self-expansion occurs when the stress of compression is removed. This allows the phase transformation from martensite back to austenite to occur, and as a result the stent expands. Materials having superelastic properties generally have at least two phases: a martensitic phase, which has a relatively low tensile strength and which is stable at relatively low temperatures, and an austenitic phase, which has a relatively high tensile strength and which can be stable at temperatures higher than the martensitic phase. Shape memory alloys undergo a transition between an austenitic phase and a martensitic phase at certain temperatures. When they are deformed while in the martensitic phase, they retain this deformation as long as they remain in the same phase, but revert to their original configuration when they are heated to a transition temperature, at which time they transform to their austenitic phase. The temperatures at which these transitions occur are affected by the nature of the alloy and the condition of the material. Nickel-titanium-based alloys (NiTi), wherein the transition temperature is slightly lower than body temperature, are preferred for the present invention. It can be desirable to have the transition temperature set at just below body temperature to insure a rapid transition from the martinsitic state to the austenitic state when the frame can be implanted in a body lumen. For example, a nitinol frame can be deformed by collapsing the frame and creating stress which causes the NiTi to reversibly change to the martensitic phase. The frame can be restrained in the deformed condition inside a delivery sheath typically to facilitate the insertion into a patient's body, with such deformation causing the isothermal phase transformation. Once within the body lumen, the restraint on the frame can be removed, thereby reducing the stress thereon so that the superelastic frame returns towards its original undeformed shape through isothermal transformation back to the austenitic phase. The shape memory effect allows a nitinol structure to be deformed to facilitate its insertion into a body lumen or cavity, and then heated within the body so that the structure returns to its original, set shape.

The recovery or transition temperature may be altered by making minor variations in the composition of the metal and in processing the material. In developing the correct composition, biological temperature compatibility must be determined in order to select the correct transition temperature. In other words, when the frame can be heated, it must not be so hot that it can be incompatible with the surrounding body tissue. Other shape memory materials may also be utilized, such as, but not limited to, irradiated memory polymers such as autocrosslinkable high density polyethylene (HDPEX). Shape memory alloys are known in the art and are discussed in, for example, “Shape Memory Alloys,” Scientific American, 281: 74-82 (November 1979), incorporated herein by reference.

An implantable frame can comprise any suitable bioabsorbable material, or combination of bioabsorbable materials. The types of bioabsorbable materials are preferably selected to provide a desired time scale for diminution in the radial strength of the frame. Variations in selected times for bioabsorption may depend on, for example, the overall health of the patient, variations in anticipated immune reactions of the patient to the implant, the site of implantation, and other clinical indicia. Bioabsorbable materials may be selected to form at least a portion of a frame so as to provide an decreased frame radial strength after a particular period of time. In certain embodiments, bioabsorption of a biomaterial in a frame can decrease the radial strength of the frame in a first direction. In some embodiments, the frame may be designed to bend radially inward in response to a pressure. The bioabsorbable material may comprise any suitable composition described with respect to the bioabsorbable coating on the removable material 12 above.

In another embodiment, the frame comprises a combination of bioabsorbable and nonabsorbable polymers. Examples of synthetic biocompatible non-bioabsorbable polymers include, but are not limited to, homopolymers and copolymers of polypropylene, polyamides, polyvinylchlorides, polysulfones, polyurethanes, polytetrafluoroethylene, ethylene vinyl acetate (EVAC), polybutylmethacrylate (PBMA) or methylmethacrylate (MMA). The frame can comprise the non-absorbable polymer in amounts from about 0.5 to about 99% of the final composition. The addition of EVAC, PBMA or methylmethacrylate increases malleability of the matrix so that the device is more plastically deformable.

The frame can include structural features, such as barbs, that maintain the frame in position following implantation in a body vessel. The art provides a wide variety of structural features that are acceptable for use in the medical device, and any suitable structural feature can be used. Furthermore, barbs can also comprise separate members attached to the frame by suitable attachment means, such as welding and bonding.

Also provided are embodiments wherein the frame comprises a means for orienting the frame within a body lumen. The frame, may be provided with marker bands at one or both of the distal and proximal ends. The marker bands (not shown) may be formed from a suitably radiopaque material. The marker bands can provide a means for orienting the medical device within a body vessel. The marker band, such as a radiopaque portion of the support member, can be identified by remote imaging methods including X-ray, ultrasound, Magnetic Resonance Imaging, fluoroscope and the like, or by detecting a signal from or corresponding to the marker band. In other embodiments, a device for delivering the medical device can comprise radiopaque indicia relating to the orientation of the support member within the body vessel. A medical device or delivery device may comprise one or more radiopaque materials to facilitate tracking and positioning of the medical device, which may be added in any fabrication method or absorbed into or sprayed onto the surface of part or all of the frame or a valve leaflet. For example, radiopaque markers can be used to identify a long axis or a short axis of a medical device within a body vessel. For instance, radiopaque material may be attached to a frame or woven into portions of the valve leaflet or other portions of the medical device. The degree of radiopacity contrast can be altered by changing the composition of the radiopaque material. For example, radiopaque material may be covalently bound to the frame or valve leaflet. Common radiopaque materials include barium sulfate, bismuth subcarbonate, and zirconium dioxide. Other radiopaque materials include: cadmium, tungsten, gold, tantalum, bismuth, platinum, iridium, iodine and rhodium.

The frame can be manufactured by any suitable approach. In one aspect, wire struts can be formed by folding a continuous member, or be joined by soldering, welding, or other methods to join ends. In another aspect, besides joining strut segments, the frame can be fabricated as a single piece of material, by stamping or cutting the frame from another sheet (e.g., with a laser), fabricating from a mold, or some similar method of producing a unitary frame. Optionally, bioabsorbable materials can be incorporated in the frame by any suitable method, including directly fabricating the frame from the bioabsorbable material, or coating one or more bioabsorbable materials onto each other or onto another material. Bioabsorbable struts can be joined to non-bioabsorbable struts by any suitable method.

Incorporation of Therapeutic Agents

In some embodiments, a therapeutic agent can be applied to or incorporated into portions of the medical device by any suitable technique, such as dipping or spray coating. One technique for applying a therapeutic agent to a medical device provides for dissolving the therapeutic agent in a suitable volatile solvent to form a solution, spraying the solution onto a portion of the medical device, and then drying the volatile solvent to deposit the therapeutic agent onto the medical device. Another technique provides for combining the therapeutic agent with a carrier material that will adhere to a portion of the medical device, such as a biodegradable polymer, and applying the therapeutic agent and the carrier material to the medical device together. For example, a poly(L-lactic acid) biodegradable polymer can be combined with a therapeutic agent to form a solution and sprayed onto the surface of the frame in the manner described by Tuch in U.S. Pat. No. 5,624,411, filed Jun. 7, 1995 and incorporated herein by reference. The frame can be formed from a porous metal material impregnated with a therapeutic agent, such as described in U.S. Pat. No. 6,240,616 to Yan, filed Apr. 15, 1997 and incorporated herein by reference. Optionally, one or more coating layers can be applied to portions of the frame to provide a sustained release of a therapeutic agent, such as described by U.S. Pat. No. 6,335,029 to Kamath, filed Dec. 3, 1998, or in U.S. patent application Ser. No. 10/414,444 by Ragheb et al., filed Apr. 14, 2003 and published as US2004/0047909A1, both of which are incorporated herein by reference. Impregnation of a valve leaflet can be accomplished using methods such as those described for impregnation of materials in U.S. Pat. No. 6,193,746 to Strecker, filed Sep. 4, 1996 and incorporated herein by reference.

Antithrombogenic therapeutic agents are particularly preferred for implantation in areas of the body that contact blood. An antithrombogenic therapeutic agent is any therapeutic agent that inhibits or prevents thrombus formation within a body vessel. Antithrombotic therapeutic agents include anticoagulants, antiplatelets, and fibrinolytics. Anticoagulants are therapeutic agents which act on any of the factors, cofactors, activated factors, or activated cofactors in the biochemical cascade and inhibit the synthesis of fibrin. Antiplatelet therapeutic agents inhibit the adhesion, activation, and aggregation of platelets, which are key components of thrombi and play an important role in thrombosis. Fibrinolytic therapeutic agents enhance the fibrinolytic cascade or otherwise aid is dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin. Other examples of antithrombotic therapeutic agents include heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other therapeutic agents such as endothelial progenitor cells or endothelial cells.

The therapeutic agent can also comprise one or more antibiotic agents. Antibiotic agents include penicillins, cephalosporins, vancomycins, aminoglycosides, quinolones, polymyxins, erythromycins, tetracyclines, chloramphenicols, clindamycins, lincomycins, sulfonamides their homologs, analogs, fragments, derivatives, pharmaceutical salts and mixtures thereof. Other therapeutic agents that can be utilized within the present invention include a wide variety of antibiotics, including antibacterial, antimicrobial, antiviral, antiprotozoal and antifungal agents.

Delivery of Medical Devices

Medical devices are preferably delivered intraluminally, for example using various types of delivery catheters, and be expanded by any suitable mechanism. For example, a medical device can be self-expanding or non-resilient. A self-expanding medical device is restrained in a compressed configuration until deployed at a point of treatment within a body vessel by releasing the medical device. Typically, a self-expanding medical device is housed within an outer sheath of a catheter delivery system, and deployed by translating the outer sheath to expose the medical device to the body vessel at the point of deployment. In contrast, a non-resilient medical device requires the application of an internal force to expand it at the target site. Typically, the expansive force can be provided by a balloon catheter, such as an angioplasty balloon for vascular procedures.

In some aspects, a frame can expand from a compressed, or unexpanded, delivery configuration to one or more radially expanded deployment configurations, for example through self-expansion or balloon expansion of the frame. In one aspect, a medical device comprises a self-expanding material. In another aspect, a medical device is expanded using a balloon catheter. The expanded frame configuration can have any suitable cross-sectional shape, including circular or elliptical. In one embodiment, the frame can be oriented along the longitudinal axis of a body vessel in the expanded or compressed configurations.

In some embodiments, the frame is self-expanding. In one aspect, a self-expanding medical device can be compressed to a delivery configuration within a retaining sheath that is part of a delivery system, such as a catheter-based system. In some aspects, a self-expanding frame can be compressed into a low-profile delivery conformation and then constrained within a delivery system for delivery to a point of treatment in the lumen of a body vessel. Upon compression, self-expanding frames can expand toward their pre-compression geometry. At the point of treatment, the self-expanding frame can be released and allowed to subsequently expand to another configuration. In one aspect, self-expanding frames preferably have an overall expansion ratio of about 1.0 up to about 4.0 times the original diameter, or more.

In some aspects, a bioabsorbable suture or sheath can be used to maintain a medical device in a compressed configuration both prior to and after deployment. As the bioabsorbable sheath or suture is degraded by the body after deployment, the medical device can expand within the body vessel. In some embodiments, a portion of the medical device can be restrained with a bioabsorbable material and another portion allowed to expand immediately upon implantation. For example, a self-expanding frame can be partially restrained by a bioabsorbable material upon deployment and later expand as the bioabsorbable material is absorbed.

Frames can also be expanded by a balloon. A medical device can be readily delivered to the desired location by mounting it on an expandable member, such as a balloon, of a delivery catheter and passing the catheter-medical device assembly through the body lumen to the implantation site. A variety of means for securing the stents to the expandable member of the catheter for delivery to the desired location arc available. It is presently preferred to compress or crimp the stent onto the unexpanded balloon. Other means to secure the stent to the balloon include providing ridges or collars on the inflatable member to restrain lateral movement, using bioabsorbable temporary adhesives, or adding a retractable sheath to cover the stent during delivery through a body lumen.

Methods for delivering a medical device as described herein are generally applicable to any suitable body vessel, such as a vein, artery, biliary duct, ureteral vessel, body passage or portion of the alimentary canal. In some embodiments, medical devices having a frame with a compressed delivery configuration with a very low profile, small collapsed diameter and great flexibility, may be able to navigate small or tortuous paths through a variety of body vessels. A low-profile medical device may also be useful in coronary arteries, carotid arteries, vascular aneurysms, and peripheral arteries and veins (e.g., renal, iliac, femoral, popliteal, subclavian, aorta, intercranial, etc.). Other nonvascular applications include gastrointestinal, duodenum, biliary ducts, esophagus, urethra, reproductive tracts, trachea, and respiratory (e.g., bronchial) ducts. These applications may or may not require a sheath covering the medical device.

Methods of Treatment

The invention also provides methods of treating a patient. In one embodiment the method comprises a step of delivering a medical device with a removable portion as described herein to a point of treatment in a body vessel, and subsequently endoluminally modifying the medical device at the point of treatment by modifying the removable portion. The delivering step can comprise delivery by surgical or by percutaneous delivery techniques known to those skilled in the art. Methods for delivering a medical device as described herein to any suitable body vessel are also provided, such as a vein, artery, biliary duct, ureteral vessel, body passage or portion of the alimentary canal.

Medical devices can be deployed in a body lumen by means appropriate to their design. The medical devices of the present invention can be adapted for deployment using conventional methods known in the art and employing percutaneous transluminal catheter devices. The medical devices are designed for deployment by any of a variety of in situ expansion means.

The medical device may be mounted onto a catheter that holds the medical device as it is delivered through the body lumen and then releases the medical device and allows it to self-expand into contact with the body lumen. This deployment is effected after the medical device has been introduced percutaneously, transported transluminally and positioned at a desired location by means of the catheter. The restraining means may comprise a removable sheath. The self-expanding medical device according to the invention may be deployed according to well-known deployment techniques for self-expanding medical devices. The medical device is positioned at the distal end of a catheter with a lubricous sleeve placed over the medical device to hold the medical device in a contracted state with a relatively small diameter. The medical device may then be implanted at the point of treatment by advancing the catheter over a guidewire to the location of the lesion and then withdrawing the sleeve from over the medical device. The medical device will automatically expand and exert pressure on the wall of the blood vessel at the site of the lesion. The catheter, sleeve, and guidewire may then be removed from the patient.

For example, the tubular body of the medical device is first positioned to surround a portion of an inflatable balloon catheter. The medical device, with the balloon catheter inside is configured at a first, collapsed diameter. The medical device and the inflatable balloon are percutaneously introduced into a body lumen, following a previously positioned guidewire in an over-the-wire angioplasty catheter system, and tracked by a fluoroscope, until the balloon portion and associated medical device are positioned within the body passageway at the point where the medical device is to be placed. Preferably, the medical device comprising a removable portion and/or the electrolytic member used to dissolve a removable portion comprise a radiopaque portion placed to allow an attending physician, using a fluoroscope, to observe the relative position of the implanted medical device and the electrolytic member. Thereafter, the balloon is inflated and the medical device is expanded by the balloon portion from the collapsed diameter to a second expanded diameter. After the medical device has been expanded to the desired final expanded diameter, the balloon is deflated and the catheter is withdrawn, leaving the medical device in place. The medical device may be covered by a removable sheath during delivery to protect both the medical device and the vessels.

The medical devices are useful for treating certain conditions, such as venous valve insufficiency, varicose veins, esophageal reflux, restenosis or atherosclerosis. In some embodiments, the invention relates to methods of treating venous valve-related conditions.

A “venous valve-related condition” is any condition presenting symptoms that can be diagnostically associated with improper function of one or more venous valves. In mammalian veins, venous valves are positioned along the length of the vessel in the form of leaflets disposed annularly along the inside wall of the vein which open to permit blood flow toward the heart and close to prevent back flow. These venous valves open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The leaflets, however, do not normally bend in the opposite direction and therefore return to a closed position to restrict or prevent blood flow in the opposite, i.e. retrograde, direction after the pressure is relieved. The leaflets, when functioning properly, extend radially inwardly toward one another such that the tips contact each other to block backflow of blood. Two examples of venous valve-related conditions are chronic venous insufficiency and varicose veins.

In the condition of venous valve insufficiency, the valve leaflets do not function properly. For example, the vein can be too large in relation to the leaflets so that the leaflets cannot come into adequate contact to prevent backflow (primary venous valve insufficiency), or as a result of clotting within the vein that thickens the leaflets (secondary venous valve insufficiency). Incompetent venous valves can result in symptoms such as swelling and varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur, for example, in the superficial venous system, such as the saphenous veins in the leg, or in the deep venous system, such as the femoral and popliteal veins extending along the back of the knee to the groin.

The varicose vein condition consists of dilatation and tortuousity of the superficial veins of the lower limb and resulting cosmetic impairment, pain and ulceration. Primary varicose veins are the result of primary incompetence of the venous valves of the superficial venous system. Secondary varicose veins occur as the result of deep venous hypertension which has damaged the valves of the perforating veins, as well as the deep venous valves. The initial defect in primary varicose veins often involves localized incompetence of a venous valve thus allowing reflux of blood from the deep venous system to the superficial venous system. This incompetence is traditionally thought to arise at the saphenofemoral junction but may also start at the perforators. Thus, gross saphenofemoral valvular dysfunction may be present in even mild varicose veins with competent distal veins. Even in the presence of incompetent perforation, occlusion of the saphenofemoral junction usually normalizes venous pressure.

The initial defect in secondary varicose veins is often incompetence of a venous valve secondary to hypertension in the deep venous system. Since this increased pressure is manifested in the deep and perforating veins, correction of one site of incompetence could clearly be insufficient as other sites of incompetence will be prone to develop. However, repair of the deep vein valves would correct the deep venous hypertension and could potentially correct the secondary valve failure. Apart from the initial defect, the pathophysiology is similar to that of varicose veins.

While many preferred embodiments discussed herein discuss implantation of a medical device in a vein, other embodiments provide for implantation within other body vessels. In another matter of terminology there are many types of body canals, blood vessels, ducts, tubes and other body passages, and the term “vessel” is meant to include all such passages.

The invention includes other embodiments within the scope of the claims, and variations of all embodiments, and is limited only by the claims made by the Applicants.

Some methods further comprise the step of implanting one or more frames attached to one or more valve members, as described herein. In some embodiments, methods of treating may also include the step of delivering a medical device to a point of treatment in a body vessel, or deploying a medical device at the point of treatment.

What is claimed:

1. A medical device comprising an endovascularly deployable frame having an electrolytically removable frame portion comprising an electrolytically removable material dissolvable by electrolysis in an electrolytic medium upon application of an electrolytically effective current through the removable material, and a valve leaflet attached to the frame.

2. The medical device of claim 1, where the valve leaflet is attached to the frame at a first attachment point and a second attachment point with a first valve leaflet tension therebetween; where removal of the electrolytically removable material decreases the first valve leaflet tension between the first attachment point and the second attachment point.

3. The medical device of claim 1, where the electrolytically removable material comprises at least one material selected from the group consisting of: stainless steel, nickel, and a nickel-titanium alloy.

4. The medical device of claim 1, wherein electrolytically effective current is about 0.01 and 2 milliamps at about 0.1 to 6 volts.

5. The medical device of claim 1, where the frame further comprises an insulating material enclosing a portion of the removable material and the electrolytically removable frame portion includes an exposed region of the electrolytically removable material that is not enclosed by the insulating material, the exposed region having a length that is between about 0.010 inch and about 0.150 inch connecting a first frame member and a second frame member.

6. The medical device of claim 1, where medical device further comprises a conducting portion in electrical conducting material in contact with the electrolytically removable frame portion.

7. The medical device of claim 6, where the medical device further comprises an insulating frame portion covering at least a portion of the conducting portion.

8. The medical device of claim 6, where the medical device further comprises a conducting portion in electrical conducting contact with the electrolytically removable frame portion.

9. The medical device of claim 1, where the frame defines a substantially cylindrical lumen and the valve leaflet is positioned within the substantially cylindrical lumen defined by the frame.

10. The medical device of claim 1, where the valve leaflet comprises a free edge moveable in response to changes in the direction of fluid flow within the body vessel.

11. The medical device of claim 1, where the removable portion is a first removable portion, where the frame has a first radial strength measured when the frame comprises the first removable portion, and the frame has a second radial strength measured when the frame does not comprise the first removable portion, where the first radial strength is greater than the second radial strength.

12. The medical device of claim 1, where the leaflet comprises at least one remodelable material.

13. The medical device of claim 1, where the frame comprises a self-expanding frame comprising a nickel-titanium alloy.

14. A medical device comprising an endovascularly implantable frame comprising
a. a first electrolytically removable portion, the frame comprising a plurality of struts and bends defining a substantially cylindrical lumen, where the frame has a first radial strength measured when the frame comprises the first removable portion, and the frame has a second radial strength measured after electrolytic removal of the first removable portion, where the first radial strength is greater than the second radial strength; and
b. at least one valve leaflet comprising a remodelable material attached to the frame at a first attachment point and a second attachment point with a first valve leaflet tension therebetween; where removal of the electrolytically removable material decreases the first valve leaflet tension between the first attachment point and the second attachment point.

15. The medical device of claim 14, where the electrolytically removable portion joins a first frame segment and a second frame segment, and further comprising a first valve leaflet attached to the frame, the first valve leaflet comprising a free edge defining a portion of a valve orifice moveable in response fluid flow contacting the first valve leaflet.

16. The medical device of claim 15, further comprising a second valve leaflet attached to the frame, the second valve leaflet comprising a free edge defining a portion of the valve orifice that is flexible to move in response to fluid flow through the body vessel, where the second valve leaflet free edge is positioned opposably to the first leaflet free edge.

17. The medical device of claim 15, wherein the first valve leaflet comprises at least one material selected from the group consisting of: an extracellular matrix material and polyurethane.

18. The medical device of claim 15, wherein the frame further comprises a flexible member joining the proximal segment and the distal segment.

19. The medical device of claim 16, where the frame comprises a nickel-titanium alloy; the medical device further comprises a conducting portion in electrical conducting contact with the electrolytically removable portion; and where the medical device further comprises an insulating frame portion covering at least a portion of the conducting portion.

20. A method of treating a subject, comprising the steps of:
a. endovascularly implanting a valve within a body vessel from a first catheter delivery system, the valve comprising an endovascularly deployable frame having an electrolytically removable frame portion comprising an electrolytically removable material dissolvable by electrolysis in an electrolytic medium upon application of an electrolytically effective current through the removable material, and a valve leaflet attached to the frame; and
b. subsequently dissolving the electrolytically removable portion by applying an electrical current from a second catheter placed within the body vessel to the electrolytically removable portion of the valve.
